(19) United States
(12) Patent Application Publication
(10) Pub. No.: US 2017/0014329 A1
(43) Pub. Date: Jan. 19, 2017

(54) WATER-BASED COLOR COMPOSITIONS

(71) Applicant: Coty Inc., New York, NY (US)

(72) Inventors: Dmitriy Yakubov, Fresh Meadows, NY (US); Beate Boutwell, Sparta, NJ (US); Virginia K. Coners, Succasunna, NJ (US); Michael L. Pavlovsky, Parsippany, NJ (US); Ann M. Sanchez, Budd Lake, NJ (US); Linda A. Bennett, Piscataway, NJ (US); Wayne M. Hoyte, Parlin, NJ (US)

(21) Appl. No.: 14/755,043

(22) Filed: Jun. 30, 2015

Related U.S. Application Data
(60) Provisional application No. 62/019,741, filed on Jul. 1, 2014.

(51) Int. Cl. A61K 8/81; A61K 8/34; A61K 8/37; A61Q 3/02; A61K 8/36; A61K 8/67; A61K 8/55; A61K 8/49; A61K 8/87; A61K 8/44

(52) U.S. Cl. CPC A61K 8/8152; A61K 8/87; A61K 8/345; A61K 8/375; A61K 8/8117; A61K 8/44; A61K 8/347; A61K 8/36; A61K 8/673; A61K 8/678; A61K 8/553; A61K 8/4953; A61K 8/4986; A61Q 3/02

(57) ABSTRACT

Inventive embodiments disclosed herein include a water-based formulation that includes silane-modified Styrene/acrylic ester copolymer in a concentration of 29.0 percent by weight; polycarbonate polyurethane dispersion in a concentration of 5.0 percent by weight; propylene glycol butyl ether in a concentration of 3.0 percent by weight; and water in a concentration of at least about 53.0 percent by weight.

### RELATED APPLICATION

This application claims the benefit of priority of U.S. Provisional Patent Application Ser. No. 62/019,741, filed on Jul. 1, 2014, which is incorporated by reference herein in its entirety.

### FIELD

Inventive embodiments disclosed herein relate to water-based color composition embodiments and to method embodiments for making the water-based color compositions. For some embodiments, the water based composition embodiments are water-based nail color embodiments.

### BACKGROUND

Color cosmetic use is traceable back through the ages. Over 3000 years ago, Chinese people stained their fingernails with a mixture of gum Arabic, gelatin, beeswax, and egg. Colors were used to signify social class. Royals wore a stain of gold or silver. Women of ancient Greece painted their faces with white lead and employed crushed mulberries as rouge.

### SUMMARY

One inventive embodiment disclosed herein includes a water-based coating formulation that includes silane-modified styrene/acrylic ester copolymer in a concentration of 29.0 percent by weight; polycarbonate polyurethane dispersion in a concentration of 5.0 percent by weight; propylene glycol butyl ether in a concentration of 3.0 percent by weight; and water in a concentration of 53.0 percent by weight.

Another embodiment includes a water-based color formulation that includes acrylates copolymer in a concentration range of 4.0 to 70.0 percent by weight; polycarbonate polyurethane dispersion in a concentration range of 1.0 percent to 45.0 percent by weight; propylene glycol butyl ether in a concentration of 3.0 by weight; and water in a concentration of 47.0 percent by weight.

Another embodiment includes a water-based color formulation that includes water in a concentration of about 60% by weight; acrylate copolymer in a concentration range of about 4.0 to 70.0% by weight; propylene glycol butyl ether in a concentration range of about 1.0 to 45.0% by weight; dipropylene glycol dibenzoate in a concentration range of 1.0 to 10.0% by weight; styrene-butyl copolymer in a concentration range of 1.0 to 25.0% by weight; colorant in a concentration range of 0.1 to 10.0% by weight; L-arginine in a concentration range of 0.01 to 1.0% by weight; phenoxyethanol in a concentration range of 0.0.1 to 1.0% by weight; acrylates copolymer in a concentration range of 0.01 to 1.5% by weight; caprylyl glycol ina concentration range 0.1 to 1.0% by weight; ammonium acrylates polymer in a concentration range of 0.1000 to 45% by weight; potassium sorbate in a concentration range of 0.01 to 1.0% by weight; L-Lysine in a concentration of 0.0001 by weight; Ca D-Patothenate in a concentration of 0.0001 percent by weight; lecitin in a concentration of 0.0001 percent by weight; D-Pantenol in a concentration of 0.0001 percent by weight; Vitamin E in a concentration of 0.0001 percent by weight; caffeine in a concentration of 0.0001 percent by weight; biotin in a concentration of 0.0001 percent by weight.

Another embodiment includes a water-based coating formulation that includes water in a concentration of at least 50% by weight; Silane-modified styrene/acrylic ester copolymer and polycarbonate polyurethane dispersion in a concentration effective for imparting hardness to a coating applied to a keratinous surface; and propylene glycol butyl ether in a concentration effective for enabling the polymers to form a continuous film when the water-based coating is applied to the keratinous surface.

Another embodiment includes a water-based coating formulation. The water-based formulation includes the following components:

| | |
|---|---|
| Water | 58.4 |
| Acrylate Copolymer | 20.9 |
| Polycarbonate polyurethane | 6.8 |
| Ethanol | 5.0 |
| Dipropylene glycol dibenzoate | 4.0 |
| PPG-2 methyl ether | 2.7 |
| Polysorbate 20 | 1.0 |
| Phenoxyethanol | 0.4 |
| DEG-90 copolymer | 0.3 |
| Caprylyl glycol | 0.2 |

Another formulation embodiment includes a water-based coating formulation that includes water in a concentration range of 58 to 59 percent by weight; Polycarbonate polyurethane in a concentration effective for fusing into a continuous film when the water-based coating is applied to the keratinous surface. The formulation also includes ethanol in a concentration effective for accelerating evaporation of water when the water-based coating is applied to the keratinous surface.

Another embodiment includes a water-based mascara. The water-based mascara includes water in a concentration of 32.2% by weight; acrylates copolymer interpolymer in a concentration of 25% by weight; stearic acid in a concentration of 5% by weight; iron oxide in a concentration of 7% by weight; and beeswax in a concentration of 7% by weight.

Another embodiment includes a water-based top coat for mascara. The top coat includes acrylate copolymer interpolymer in a concentration of 45% by weight; polycarbonate polyurethane in a concentration of 21% by weight; polycarbonate urethane in a concentration of 7% by weight; ethanol in a concentration of 5% by weight; acrylamide/sodium acryloyldimethyltaurate copolymer and isohexadecane and polysorbate 80 in a concentration of 5.5% by weight; and black iron oxide in a concentration of 7.5% by weight.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

A first formulation embodiment is a water-based nail color formulation, which is as follows:

### Formulation 1

| Ingredient | Concentration |
|---|---|
| Water | 60.3997 |
| Acrylate copolymer | 24.6000 |
| Propylene glycol butyl ether | 4.0000 |
| Dipropylene glycol dibenzoate | 4.0000 |
| Styrene-butyl copolymer | 3.9000 |
| Colorant | 1.5000 |
| L-arginine | 0.5000 |
| Phenoxyethanol | 0.4000 |
| Acrylates copolymer | 0.3000 |
| Caprylyl glycol | 0.2000 |
| Ammonium acrylates polymer | 0.1000 |
| Potassium sorbate | 0.1000 |
| L-Lysine | 0.0001 |
| Ca D-Pantothenate | 0.0001 |
| Lecitin | 0.0001 |
| D-Pantenol | 0.0001 |
| Vitamin E | 0.0001 |
| Caffeine | 0.0001 |
| Biotin | 0.0001 |
| Total | 100.0000 |

Concentrations are presented as a percent by weight. While specific concentrations are presented in the table for formulation 1, the following concentration ranges produce acceptable water-based nail color formulations: acrylate copolymer of 4.0 to 70.0 weight percent; propylene glycol butyl ether of 1.0 to 45.0 percent by weight; dipropylene glycol dibenzoate of 1.0 to 10.0 percent by weight; styrene butyl copolymer of 1 to 25.0 percent by weight; ethanol in a concentration of 1.0 to 10.0 percent by weight; colorant in a concentration of 0.01 to 10.0 percent by weight; L-arginine in a concentration of 0.01 to 1.0 percent by weight; phenoxyethanol in a concentration of 0.1 to 1.0 percent by weight; acrylates copolymer in a concentration of 0.01 to 1.5 percent by weight; caprylyl glycol in a concentration of 0.01 to 1.0 percent by weight; ammonium acrylates polymer in a concentration of 1.0 to 45.0 percent by weight; and potassium sorbate of 0.01 to 1.0 percent by weight. Remaining ingredients shown in the table for formulation 1 have the same concentrations shown in the table.

A polymer combination of styrene-butyl copolymer, acrylates copolymer and ammonium acrylates copolymer acts to make a high gloss, long retention coating or polish for a keratinous material. In one embodiment, the polymer combination is used to impart long wear to a nail polish. The polymer combination is suspended in the water component. The propylene glycol butyl ether acts to enable the polymers to form a continuous film when applied to a keratinous material and form a coating. Other components in the formulation aid in imparting a high gloss and long retention coating.

Another embodiment includes a water-based coating, comprising: Water in a concentration of at least 50% by weight; silane-modified Styrene/Acrylic Ester Copolymer and Polycarbonate polyurethane Dispersion in a concentration effective for imparting hardness to a coating applied to a keratinous surface; and propylene Glycol Butyl Ether in a concentration effective for enabling the polymers to form a continuous film when the water-based coating is applied to the keratinous surface.

Another embodiment is a formulation for a water-based top coat for application over a coating such as are disclosed as Formulations 1, 3 and 4. The water-based topcoat formulation is as follows:

### Formulation 2

| Ingredient | Concentration |
|---|---|
| Water | 58.4 |
| Acrylate copolymer | 20.9 |
| Polycarbonate polyurethane | 6.8 |
| Ethanol | 5.0 |
| Dipropylene glycol dibenzoate | 4.0 |
| PPG-2 methyl ether | 2.7 |
| Polysorbate 20 | 1.0 |
| Phenoxyethanol | 0.4 |
| DEG-90 copolymer | 0.3 |
| Caprylyl glycol | 0.2 |

Concentrations presented herein refer to a percent by weight of the formulations. While specific concentrations are shown in the table for Formulation 2, it is understood that concentrations within ranges for particular ingredients may be employed. In particular, acrylate copolymer has an acceptable concentration range of 4.0 to 70.0 percent by weight; polycarbonate polyurethane has a concentration range of 1.- to 45.0 percent by weight; ethanol has a concentration range of 1.0 to 10.0 percent by weight; dipropylene glycol dibenzoate has a concentration range of 1.0 to 10.0 percent by weight; PPG-2 methyl ether has a concentration range of 1.0 to 10.0 percent by weight; Polysorbate 20 has a concentration range of 0.1 to 3.0 percent by weight; phenoxyethanol has a concentration range of 0.1 to 1.0 percent by weight; DEG-90 copolymer has a concentration range of 0.01 to 1.0 percent by weight; and caprylyl glycol has a concentration range of 0.01 to 1.0 percent by weight.

For Formulation 2, the polycarbonate polyurethane and its modulators form a second coating when applied to a first coating on a keratinous material, such as a nail. Water acts as a carrier for the polycarbonate polyurethane and modulators of the polycarbonate polyurethane. PPG-2 methyl ether enables the polymers to form a continuous film when the formulation is applied to the coating on the keratinous surface. Ethanol is added in a concentration effective for accelerating evaporation of water when the water-based coating is applied to the keratinous surface.

The top coat, Formulation 2, is applied on a water based nail color coating, presented above as Formulation 1 or on coatings made with Formulations 3 and 4 below, once the coating formed by the color formulation 1, 3 or 4 is dry. The color formulation 2 provides for a longer wear as well as a very high gloss, and gloss retention. The gloss remains high through the length of wear of the nail polish top coated with the top coat formulation. The water-based color formulation embodiments disclosed herein provide good adhesion to a keratinous surface such as a nail surface, as well as flexibility, chip resistance, extended wear, and good gloss.

Polymers are selected for the formulation embodiments disclosed herein so that once a coating is applied to a keratinous surface, the water in the formulation evaporates and the polymers, with the help of a coalescent, such as glycol ether, form a film which has a good adhesion to the keratinous surface as well as all of the other properties for the nail color formulation embodiments disclosed herein. For some embodiments, the glycol ether is Dowanol PnB or

### Dowanol TPM, Manufactured by Dow Chemical.

The top coat, formulation 2, is applied on top of the applied dry nail color formulation coating. While the formulations 1, 3, and 4 are disclosed herein, it is understood that other nail coating embodiments are suitable for use with the topcoat made by formulation 2. The film of the top coat develops an excellent adhesion to the nail color, protects it for longer wear, and provides great gloss retention.

In another formulation embodiment, Formulation 3, the water-based cosmetic formulation is a nail color formulation that includes water in a concentration range of 50.0 to 55.0% by weight of the formulation. Formulation 3 is shown below. Water is present in a concentration of 53% by weight.

### Formulation 3

| | |
|---|---|
| Silane-modified Styrene/Acrylic Ester Copolymer | 29.0 |
| Polycarbonate Polyurethane Dispersion | 15.0 |
| Propylene Glycol Butyl Ether | 3.0 |
| Water | 53.0 |
| Total | 100.0 |

### Formulation 4

| | |
|---|---|
| Ammonium Acrylates Copolymer | 43.0 |
| Polycarbonate Polyurethane Dispersion | 8.0 |
| Propylene Glycol Butyl Ether | 2.0 |
| Water | 47.0 |
| Total | 100.0 |

Formulation 3 is a microemulsion usable for a variety of cosmetics. For formulation 3, polymerization in oil in water microemulsions produces ultrafine nanolatex particles in a narrow size distribution, such as 10 to 100 nanometers. For all formulations disclosed herein, the silane-modified styrene/acrylic ester copolymer is such as is disclosed in US 20150166708 and U.S. Pat. No. 7,723,426. For all formulation embodiments disclosed herein, the polycarbonate-polyurethane dispersion is such as is Alberdingk U6150. The term, ammonium acrylates copolymer, refers to a copolymer of more than one acrylate, including at least one acrylic/methacrylic acid component capable of forming a salt such as an ammonium salt.

Formulations 3 and 4, when formulated to a nail coating, impart a good adhesion to nail, flexibility, chip resistance, extended wear, good gloss.

The formulations 1-4 are usable with one or more additives such as rheology modifiers, surfactants, defoamers, slip additives, plasticizers and pigments.

The formulation embodiments disclosed herein are usable to make coatings that display long-term biostability. For some embodiments, the coatings act as primer layers for attachment of other layers or for bio-active agents. Because the coating formulations are aqueous-based, the coatings are less hazardous than solvent-based coatings. Moreover, the coating formulation embodiments are highly versatile and are efficiently applied to a wide range of surfaces, such as keratinous surfaces.

In addition to keratinous surfaces, the formulations 1, 2, 3 and 4 are usable in formulations for cosmetics such as lipsticks, lip gloss, blush, eye liner and eyeshadow.

Another embodiment includes a water-based mascara having water in a concentration of 31.2% by weight; acrylates copolymer, interpolymer, in a concentration of 25% by weight; stearic acid in a concentration of 5% by weight; iron oxide in a concentration of 7% by weight; and beeswax in a concentration of 7% by weight.

One water-based tattoo mascara formulation is as follows:

### Formula 5:

Water based Tattoo Mascara

| INCI | % |
|---|---|
| Stearic Acid | 5.0 |
| Carnuba Wax | 3.0 |
| Beeswax | 7.0 |
| Hydrogenated polyisobutene | 3.0 |
| Glyceryl stearate | 1.5 |
| Iron Oxide | 7.0 |
| Candelilla wax | 0.2 |
| water | 31.15 |
| Disodium EDTA | 0.2 |
| Phenoxyethanol | 0.5 |
| Caprylyl Glycol | 0.5 |
| Nylon fibers 2 mm | 1.0 |
| simethicone | 0.25 |
| Acrylates Copolyer - Interpolymer | 24.9 |
| Propylene Glycol Butyl Ether | 1.0 |
| Styrene Butyl Copolymer | 3.0 |
| Dipropylene Glycol Dibenzoate | 1.0 |
| Glycerin | 4.0 |
| Polysorbate 80 | 3.0 |
| Sodium Dehydroacetate | 0.3 |
| Microcrystalline Cellulose | 2.0 |
| Hectorite | 0.5 |
| Total = | 100.00 |

Another formulation embodiment is for a water-based top coat for mascara or for a tattoo liquid eyeliner. The formulation includes a water-based top coat for mascara, having water in a concentration of 45% by weight; acrylate copolymer, interpolymer, in a concentration of 21% by weight; polycarbonate polyurethane in a concentration of 7% by weight; ethanol in a concentration of 5% by weight; acrylamide/sodium acryloyldimethyltaurate copolymer and isohexadecane and polysorbate 80 in a concentration of 5.5% by weight; and black iron oxide in a concentration of 7.5% by weight.

The formulation is as follows:

### Formula 6:

Water based Tattoo Top Coat for Mascara or Tattoo liquid Eyeliner

| INCI | % |
|---|---|
| water | 45.0 |
| Acrylate Copolymer - Interpolymer | 21.0 |
| Polycarbonate Polyurethane | 7.0 |
| Dipropylene Glycol Dibenzoate | 1.0 |
| Ethanol | 5.0 |
| Disodium edta | 0.2 |
| Sodium Dehydroacetate | 0.3 |
| Polysorbate 80 | 3.0 |
| Butylene Glycol | 4.0 |
| Black iron oxide | 7.5 |
| Acrylamide/Sodium Acryloyldimethyltaurate Copolymer | 5.5 |
| (and) Isohexadecane (and) Polysorbate 80 | |
| Phenoxyethanol | 0.5 |
| Total = | 100.0 |

The formulations 5 and 6 are also usable with one or more additives such as rheology modifiers, surfactants, defoamers, slip additives, plasticizers and pigments.

All of the formulation embodiments disclosed herein are usable to make coatings that display long-term biostability. For some embodiments, the coatings act as primer layers for attachment of other layers or for bio-active agents. Because the coating formulations are aqueous-based, the coatings are less hazardous than solvent-based coatings. Moreover, the coating formulation embodiments are highly versatile and are efficiently applied to a wide range of surfaces, such as keratinous surfaces.

For all formulations, by “interpolymer” is meant an acrylic copolymer in which two monomer units are so intimately distributed in the polymer molecule that the substance is essentially homogeneous in chemical composition. For some embodiments, the acrylic polymer, Interpolymer, is a SYNTRAN acrylic polymer dispersion.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A water-based nail cover formulation, comprising:
silane-modified styrene/acrylic ester copolymer;
polycarbonate polyurethane dispersion;
propylene glycol butyl ether; and
water, wherein the formulation comprises particles in a size distribution of 10 to 100 nanometers.

2. The water-based formulation of claim 1, further comprising one or more of rheology modifiers, surfactants, defoamers, slip additives, plasticizers and pigments.

3. The water-based formulation of claim 1, further comprising vitamins.

4. The water-based formulation of claim 1, which is a keratinous coating.

5. The water-based formulation of claim 1, which is a coating that coats cosmetic particles.

6.-9. (canceled)
